(19) United States
(12) Patent Application Publication
(10) Pub. No.: US 2014/0303189 A1
(43) Pub. Date: Oct. 9, 2014

(54) PATCH

(71) Applicants: Masaoki Goto, Kagawa (JP); Atsuyo Hamada, Kagawa (JP); Mitsuji Akazawa, Kagawa (JP); Keiko Yamasaki, Kagawa (JP); Shoji Miyaji, Kagawa (JP)

(72) Inventors: Masaoki Goto, Kagawa (JP); Atsuyo Hamada, Kagawa (JP); Mitsuji Akazawa, Kagawa (JP); Keiko Yamasaki, Kagawa (JP); Shoji Miyaji, Kagawa (JP)

(73) Assignee: KM TRANSDERM LTD., Osaka-shi, Osaka (JP)

(21) Appl. No.: 14/239,475

(22) PCT Filed: Aug. 17, 2012
(86) PCT No.: PCT/JP2012/070933

§ 371 Date: May 23, 2014

(30) Foreign Application Priority Data
Aug. 19, 2011 (JP) 2011-179988

(51) Int. Cl. A61K 9/70; A61K 31/519

(52) U.S. Cl. CPC A61K 9/7053; A61K 31/519
USPC 514/259.41

(57) ABSTRACT

The invention provides a patch containing a support and, formed thereon, an adhesive layer retaining a drug. The adhesive layer contains a thermoplastic elastomer, a liquid component in an amount exceeding 300 parts by weight relative to 100 parts by weight of the elastomer, and risperidone or a salt thereof, and optionally a tackifier of not more than 10 wt %.

### TECHNICAL FIELD

The present invention relates to a patch containing risperidone or a salt thereof. More particularly, the present invention relates to a patch showing high skin permeability of risperidone or a salt thereof and good transdermal absorbability.

### BACKGROUND ART

When transdermal absorption of a drug is desired, the drug is added to an adhesive matrix and the like and a patch is formed. In recent years, a tape agent more superior in the adhesiveness is more often used than cataplasm containing a large amount of water as a constituent component in the patch. As the adhesive matrix for this tape agent, lipophilic adhesive matrices such as rubber, acrylic, silicone series and the like are used. Of these, rubber adhesive matrices are widely used since they permit easy blending of additive as compared to other adhesive matrices (patent documents 1-3).

However, problems in that sufficient releasability of the drug cannot be ensured, skin irritation caused by a tackifier generally added to a patch is developed and the like have also been pointed out for a patch using a rubber adhesive matrices.

Incidentally, schizophrenia is a mental disorder characterized by basic and characteristic twisting of thought and perception and inappropriate and dulled emotion, and characteristically shows positive symptoms such as delusion, hallucination, paranoia and the like, and negative symptoms such as social withdrawal and blunted affect. The sign of schizophrenia generally manifests itself between 16 and 25 years of age, and the disease is developed at the rate of one out of 100 people in the world. It is a disease more often seen than Alzheimer's disease, multiple sclerosis, insulin dependent diabetes and muscular dystrophy. It is known that early medical examination and treatment result in the recovery therefrom or remarkable improvement of the symptoms, and early therapeutic intervention can avoid costly hospitalization and the like.

As a pharmaceutical agent effective for the treatment of schizophrenia, risperidone, i.e., 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidine-4-one, is known (patent document 4). The effect of risperidone on schizophrenia is considered to be afforded by controlling the central nervous system mainly based on the dopamine D₂ receptor antagonistic action and the serotonin 5-HT₂ receptor antagonistic action, and risperidone is currently used widely as a therapeutic drug for schizophrenia.

Risperidone is conventionally administered orally, and marketed in the dosage forms of tablet, fine granules, liquid and the like. However, when symptoms have progressed in schizophrenia patients, oral administration of a pharmaceutical agent sometimes becomes difficult. Therefore, administration of antipsychotic agents such as risperidone and the like by a method other than oral administration is desired.

Thus, exploitation of a new administration method of risperidone has been tried, and the development of a transdermal therapeutic system (TTS) other than oral administration has been tried (patent documents 5, 6). Patent document 5 discloses a medical patch for transdermal administration, which contains fatty acids, fatty acid monoglyceride and the like as a skin penetration promoter, and patent document 6 discloses a preparation for transdermal application containing alcohols, polyvalent alcohol, higher alkane, higher fatty acid, higher fatty acid ester and the like as a transdermal absorption promoter. However, these transdermal absorption preparations have not been put to practical use, since the release of risperidone from the preparation, skin permeability and the like are not sufficient and the effect of TTS has not been exhibited sufficiently.

Also, improvement of the transdermal absorbability of basic drugs such as risperidone and the like has been tried by adding, to a patch, an organic acid or a salt thereof as a melting point depressant for the drug (patent documents 7, 8). However, addition of an organic acid or a salt thereof may increase the skin irritation caused by the patch.

### DOCUMENT LIST

### Patent Documents

patent document 1: JP-A-2001-302502
patent document 2: JP-A-9-291028
patent document 3: JP-A-10-316559
patent document 4: JP-B-6-13511
patent document 5: JP-A-11-503138
patent document 6: JP-A-2008-169145
patent document 7: WO2006/093139
patent document 8: JP-A-2006-169238

### SUMMARY OF THE INVENTION

### Problems to be Solved by the Invention

An object of the present invention is to provide a patch showing sufficient adhesiveness, low skin irritation, good skin permeability of risperidone or a salt thereof, and sufficient transdermal absorbability.

### Means of Solving the Problems

The present inventors have conducted intensive studies in an attempt to solve the aforementioned problems and found that the skin irritation can be reduced while affording sufficient adhesiveness, even if a tackifier is not contained, by using, as an adhesive matrix, a thermoplastic elastomer and a large amount of a liquid component relative to the elastomer and, when risperidone or a salt thereof is added, a patch of risperidone or a salt thereof exhibiting good skin permeability and sufficient transdermal absorbability can be obtained, which resulted in the completion of the present invention.

Accordingly, the present invention relates to the following (1)-(11).

(1) A patch comprising an adhesive layer retaining a drug formed on a support, wherein the aforementioned adhesive layer comprises a thermoplastic elastomer, a liquid component in an amount exceeding 300 parts by weight relative to 100 parts by weight of the elastomer, and risperidone or a salt thereof, and optionally comprises a tackifier, wherein the content thereof in the adhesive layer is not more than 10 wt %.
(2) The patch of the above-mentioned (1), wherein the liquid component comprises one or more kinds selected from the group consisting of a non-volatile hydrocarbon oil, a liquid surfactant, an ester solvent, an alcohol solvent and an amide solvent.
(3) The patch of the above-mentioned (1), wherein the liquid component comprises a non-volatile hydrocarbon oil and a liquid component other than the non-volatile hydrocarbon oil.
(4) The patch of the above-mentioned (2) or (3), wherein the non-volatile hydrocarbon oil is liquid paraffin.
(5) The patch of the above-mentioned (3), wherein the liquid component other than the non-volatile hydrocarbon oil comprises one or more kinds selected from the group consisting of a liquid surfactant, an ester solvent, an alcohol solvent and an amide solvent.
(6) The patch of the above-mentioned (3), wherein the liquid component other than the non-volatile hydrocarbon oil comprises one or more kinds of ester solvents, and one or more kinds selected from the group consisting of an alcohol solvent and an amide solvent.
(7) The patch of the above-mentioned (2) or (5), wherein the liquid surfactant is a liquid sorbitan fatty acid ester.
(8) The patch of any of the above-mentioned (1)-(7), wherein the content of the liquid component in the adhesive layer is not less than 60 wt %.
(9) The patch of any of the above-mentioned (1)-(8), wherein the thermoplastic elastomer is a styrene block copolymer.
(10) The patch of the above-mentioned (9), wherein the styrene block copolymer is one or more kinds selected from the group consisting of a styrene-isoprene-styrene block copolymer and a styrene-isoprene block copolymer.
(11) The patch of any of the above-mentioned (1)-(10), wherein the adhesive layer is free of a tackifier.

### Effect of the Invention

The patch of the present invention shows sufficient adhesiveness when adhered to the skin, reduced skin irritation, good skin permeability of risperidone or a salt thereof, and superior transdermal absorbability.

### BRIEF DESCRIPTION OF THE DRAWINGS

FIG. 1 is a graph showing the skin permeability of risperidone in the patches of Examples 1-3 of the present invention.

FIG. 2 is a graph showing the skin permeability of risperidone in the patch of Example 4 and the patch of Comparative Example 2 of the present invention.

FIG. 3 is a graph showing the transdermal absorbability of the patch of Example 3 of the present invention.

### DESCRIPTION OF EMBODIMENTS

The patch of the present invention comprises an adhesive layer retaining a drug, which is formed on a support, wherein the aforementioned adhesive layer comprises a thermoplastic elastomer, a liquid component in an amount exceeding 300 parts by weight relative to 100 parts by weight of the elastomer, and risperidone or a salt thereof, and optionally comprises a tackifier, and its content in the adhesive layer is not more than 10 wt %.

The “thermoplastic elastomer” used in the present invention is an elastomer having thermoplasticity that shows flowability since it softens by adding heat and returns to a rubbery elastic body by cooling, and various thermoplastic elastomers of urethane, acrylic, styrene, olefin series and the like can be mentioned. Of these, styrene thermoplastic elastomers, particularly, a styrene block copolymer, are preferably used to simultaneously impart sufficient adhesiveness and reduce skin irritation, which is the object of the present invention.

Specific examples of the styrene block copolymer include a styrene-butadiene block copolymer, a styrene-butadiene-styrene block copolymer, a styrene-isoprene block copolymer, a styrene-isoprene-styrene block copolymer, a styrene-ethylene/butylene block copolymer, a styrene-ethylene/butylene-styrene block copolymer, a styrene-ethylene/propylene block copolymer, a styrene-ethylene/propylene-styrene block copolymer, a styrene-isobutylene block copolymer, a styrene-isobutylene-styrene block copolymer and the like. In the above, the “ethylene/butylene” shows a copolymer block of ethylene and butylene, and the “ethylene/propylene” shows a copolymer block of ethylene and propylene. Only one kind of these styrene block copolymers may be used or two or more kinds thereof may be used in combination.

Among the above-mentioned styrene block copolymers, one or more kinds selected from the group consisting of a styrene-isoprene-styrene block copolymer and a styrene-isoprene block copolymer are particularly preferably used, to simultaneously achieve adhesiveness and low skin irritation, and in view of the availability and handlability of the patch products.

For the object of the present invention, a styrene-isoprene-styrene block copolymer preferably has a content of the styrene unit in the copolymer of 5 wt %-60 wt %, more preferably 10 wt %-50 wt %. In addition, it preferably has a weight average molecular weight as measured by gel filtration chromatography of 20,000-500,000, more preferably 30,000-300,000. As the styrene-isoprene block copolymer, one having a content of the styrene unit in the copolymer of 5 wt %-50 wt %, more preferably 10 wt %-40 wt %. In addition, it preferably has a weight average molecular weight as measured by gel filtration chromatography of 10,000-500,000, more preferably 20,000-300,000.

As the styrene-isoprene-styrene block copolymer or styrene-isoprene block copolymer, a copolymer produced by a method known per se can be used, or a commercially available product satisfying the above-mentioned property, for example, “KRATON D” (manufactured by KRATON POLYMERS), “JSR SIS” (manufactured by JSR) and the like can also be used.

In the present invention, the “liquid component” is liquid at ambient temperature, does not volatilize during production and storage, and adhesion, remains in the adhesive layer, and added to a patch as a plasticizer or softening agent, or a dispersing agent and/or a transdermal absorption promoter for risperidone or a salt thereof. Here, the “ambient temperature” means 15° C.-25° C. Accordingly, the aforementioned liquid component is a substance having a melting point of lower than the ambient temperature, and a boiling point of preferably not less than 150° C. and more preferably not less than 170° C. In the present invention, a substance having a certain level of viscosity can also be used. As the “liquid component” of the present invention, one having a viscosity at 25° C. of 0.01 mPa·s-1,000,000 mPa·s can be used.

In the present invention, examples of the “liquid component” include non-volatile hydrocarbons, liquid surfactants, ester solvents, alcohol solvents, amide solvents and the like.

As the non-volatile hydrocarbons, a chain saturated hydrocarbon having a carbon number of about 20-40 or a chain unsaturated hydrocarbon having a carbon number of about 20-40 is preferable and, for example, liquid paraffin, squalene, squalane, pristane and the like can be mentioned. Particularly, liquid paraffin is more preferable. Liquid paraffin is a mixture of alkanes which are colorless odorless and liquid, and have a carbon number of not less than 20. In the present invention, one that satisfies the standards of the Japanese Pharmacopoeia, US Pharmacopeia and the like, and the like can be preferably used.

The “liquid surfactant” refers to a surfactant which is liquid at ambient temperature.

Examples of the surfactant include nonionic surfactants which are liquid at ambient temperature, for example, polyoxyethylene fatty acid ester which is liquid at ambient temperature such as polyoxyethylene(10)monolaurate and the like, polyoxyethylene sorbit fatty acid ester which is liquid at ambient temperature such as polyoxyethylene(6-60)sorbit tetraoleate and the like, polyoxyethylene sorbitan fatty acid ester which is liquid at ambient temperature such as polyoxyethylene(20)sorbitan monooleate, polyoxyethylene(20)sorbitan monolaurate, polyoxyethylene(20)sorbitan monopalmitate and the like, sorbitan fatty acid ester which is liquid at ambient temperature such as sorbitan monolaurate, sorbitan monooleate, sorbitan sesquioleate, sorbitan trioleate and the like, glycerol fatty acid ester which is liquid at ambient temperature such as glycerol monooleate, polyoxyethylene(3-10)castor oil derivative, polyoxyethylene(5-20)hydrogenated castor oil and the like, polyoxyethylene higher aliphatic alcohol ether which is liquid at ambient temperature such as polyoxyethylene(2-9)lauryl ether, polyoxyethylene(2-7)oleyl ether and the like, polyoxyethylene alkylphenyl ether which is liquid at ambient temperature such as polyoxyethylene(2-15)nonylphenyl ether and the like, polyoxyethylene polyoxypropylene copolymer which is liquid at ambient temperature such as pluronic L-31, pluronic L-44 and the like, and the like can be mentioned.

Of these, sorbitan fatty acid ester which is liquid at ambient temperature is more preferable, and sorbitan mono laurate is particularly preferable, to increase the transdermal absorbability.

In the present invention, the content of the surfactant in the adhesive layer is preferably 0.01 wt %-10 wt %, more preferably 0.1 wt %-5 wt %, of the total weight of the adhesive layer.

Examples of the ester solvent include ester of long chain fatty acid and monovalent aliphatic alcohol, middle chain fatty acid triglyceride, ester of polyvalent carboxylic acid and monovalent aliphatic alcohol, carbonate and the like.

Examples of the ester of long chain fatty acid and monovalent aliphatic alcohol include an ester of long chain saturated fatty acid having a carbon number of 12-20 and monovalent aliphatic alcohol having a carbon number of 1-20, which is liquid at ambient temperature, is preferable and, for example, myristic acid ester which is liquid at ambient temperature such as ethyl myristate, isopropyl myristate, myristic acid octyldodecyl ester and the like, palmitic acid ester which is liquid at ambient temperature such as ethyl palmitate, isopropyl palmitate, palmitic acid isostearyl ester and the like, stearic acid ester which is liquid at ambient temperature such as stearic acid isopropyl ester and the like, and the like. In addition, an ester of long-chain unsaturated fatty acid having a carbon number of 12-20 and monovalent aliphatic alcohol having a carbon number of 1-20 can also be preferably used. For example, oleic acid ester which is liquid at ambient temperature such as ethyl oleate, decyl oleate, oleyl oleate and the like, linoleic acid ester which is liquid at ambient temperature such as ethyl linoleate, linoleic acid isopropyl ester and the like, and the like can be mentioned.

The middle chain fatty acid triglyceride is a triglyceride composed of a fatty acid having a carbon number of about 6-12 such as caproic acid, caprylic acid, capric acid, lauric acid and the like and glycerol. In the present invention, caprylic acid triglyceride, a triglyceride mixture of caprylic acid and capric acid, a triglyceride mixture of caprylic acid, capric acid and lauric acid, which is liquid at ambient temperature, and the like can be used. In addition, fat or oil containing a large amount of these, which is liquid at ambient temperature, can also be used. Examples of such fat and oil include earthnut oil, olive oil, castor oil and the like.

In the present invention, moreover, a commercially available product marketed as a pharmaceutical product can also be used as middle chain fatty acid triglyceride which is liquid at ambient temperature, or fat or oil containing middle chain fatty acid triglyceride, which is liquid at ambient temperature.

Examples of the ester of polyvalent carboxylic acid and monovalent aliphatic alcohol include a diester of dicarboxylic acid having a carbon number of 2-12 and monovalent aliphatic alcohol having a carbon number of 1-20, which is liquid at ambient temperature, for example, an adipic acid diester such as adipic acid diethyl ester, adipic acid diisopropyl ester and the like, which is liquid at ambient temperature, a sebacic acid diester such as sebacic acid diethyl ester, sebacic acid diisopropyl ester, sebacic acid dioctyldodecyl ester and the like, which is liquid at ambient temperature, and the like.

As carbonate, a cyclic carbonate of carbonic acid and diol having a carbon number of 2-10, for example, ethylene carbonate, propylene carbonate, vinylene carbonate and the like can be mentioned, and propylene carbonate is preferable.

Among the above-mentioned ester solvents, myristic acid ester, a middle chain fatty acid triglyceride mixture, sebacic acid ester and carbonate are preferable, and isopropyl myristate, a triglyceride mixture of caprylic acid and capric acid, sebacic acid diethyl ester and propylene carbonate are more preferable.

Examples of the alcohol solvent include a higher saturated aliphatic alcohol having a carbon number of about 12-20, which is liquid at ambient temperature, such as lauryl alcohol, isostearyl alcohol, 2-octyl dodecanol and the like; a higher unsaturated aliphatic alcohol having a carbon number of about 12-20, which is liquid at ambient temperature, such as oleyl alcohol and the like; polyvalent alcohol which is liquid at ambient temperature such as ethylene glycol, propylene glycol, glycerol, 1,3-butanediol, polyethylene glycol having a molecular weight of about 100-600 and the like, and the like.

Particularly, a polyvalent alcohol which is liquid at ambient temperature, such as ethylene glycol, propylene glycol, glycerol, 1,3-butanediol, polyethylene glycol and the like is preferable, and a diol which is liquid at ambient temperature, such as ethylene glycol, propylene glycol, 1,3-butanediol, polyethylene glycol having a molecular weight of about 100-600, and the like are more preferable.

As the amide solvent, for example, pyrrolidone such as N-methyl-2-pyrrolidone, 2-pyrrolidone and the like; imidazolidinone such as 1,3-dimethyl-2-imidazolidinone and the like; alkanamide such as formamide, N-methylformamide, N,N-dimethylformamide, N-methylacetamide, N,N-dimethylacetamide, N-methylpropanamide and the like; phosphoramide such as hexamethylphosphoric triamide and the like, and the like can be mentioned.

Among the above-mentioned amide solvents, N-methyl-2-pyrrolidone, N,N-dimethylformamide, and N,N-dimethylacetamide are preferable, and N-methyl-2-pyrrolidone is more preferable.

In the patch of the present invention, the non-volatile hydrocarbon oil, particularly liquid paraffin, is preferably contained as a liquid component. In consideration of the skin permeability of risperidone or a salt thereof, the non-volatile hydrocarbon oil is preferably contained together with other liquid component. The content of the non-volatile hydrocarbon oil such as liquid paraffin and the like in the adhesive layer is generally 12 wt %-92.5 wt %, preferably 15 wt %-90 wt %, more preferably 20 wt %-80 wt %. In addition, the content of the non-volatile hydrocarbon oil in the total weight of the liquid component is preferably 20 wt %-97 wt %, more preferably 30 wt %-90 wt %.

From the aspect of enhancement of the dispersibility in the adhesive layer and transdermal absorbability of risperidone or a salt thereof, one or more kinds selected from the group consisting of a liquid surfactant, an ester solvent, an alcohol solvent and an amide solvent is/are preferably contained together with a non-volatile hydrocarbon oil such as liquid paraffin and the like as a liquid component. Of these, one or more kinds of ester solvent, and one or more kinds selected from the group consisting of an alcohol solvent and an amide solvent are preferably contained in combination to improve transdermal absorbability, and particularly, the weight ratio of (one or more kinds of ester solvent):(one or more kinds selected from the group consisting of alcohol solvent and amide solvent) is preferably set to 1:1-1:4 to enhance the transdermal absorbability-improving effect.

The content of one or more kinds selected from the group consisting of a liquid surfactant, an ester solvent, an alcohol solvent and an amide solvent as the total amount thereof is preferably 3 wt %-80 wt %, more preferably 10 wt %-70 wt %, relative to the total weight of the liquid component, and preferably 2 wt %-75 wt %, more preferably 7 wt %-65 wt %, relative to the total weight of the adhesive layer.

As mentioned above, the patch of the present invention contains the above-mentioned liquid component in a total amount exceeding 300 parts by weight per 100 parts by weight of the thermoplastic elastomer. When the content of the liquid component per 100 parts by weight of the thermoplastic elastomer is not more than 300 parts by weight, sufficient adhesiveness cannot be obtained. On the other hand, when the content of the liquid component relative to the thermoplastic elastomer is too much, the shape of the adhesive layer is generally difficult to maintain. Therefore, the upper limit of the content of the liquid component does not generally exceed 1500 parts by weight per 100 parts by weight of the thermoplastic elastomer. The content of the liquid component per 100 parts by weight of the thermoplastic elastomer is preferably 320 parts by weight-1000 parts by weight, more preferably 340 parts by weight-700 parts by weight.

On the other hand, when the content of the thermoplastic elastomer in the adhesive layer is too small, the shape of the adhesive layer is generally difficult to maintain, and when it is too much, the adhesiveness becomes insufficient. Therefore, the thermoplastic elastomer content of the adhesive layer in the patch of the present invention is generally 5 wt %-24.7 wt %, preferably 8 wt %-24 wt %, more preferably 10 wt %-23 wt %. The content of the above-mentioned liquid component in the adhesive layer is generally 60 wt %-94.9 wt %, preferably 65 wt %-91.9 wt %, more preferably 70 wt %-89.9 wt %, further preferably 72 wt %-88 wt %, particularly preferably 75 wt %-85 wt %.

In the patch of the present invention, good adhesiveness can be exhibited when a thermoplastic elastomer and a liquid component are contained at the above-mentioned contents and content ratio in the adhesive layer, and the adhesive layer may also contain a tackifier as necessary.

Here, the tackifier is a resin generally used widely for conferring adhesiveness in the field of patch and, for example, rosin resin, polyterpene resin, coumarone-indene resin, petroleum resin, terpene-phenol resin, alicyclic saturated hydrocarbon resin and the like can be mentioned.

However, to reduce skin irritation and the like, the content of the tackifier in the adhesive layer in the present invention is not more than 10 wt % relative to the total weight of the adhesive layer. This content is preferably not more than 5 wt %, more preferably not more than 2 wt %, further preferably not more than 1 wt %, and most preferably free of a tackifier. The content of the tackifier is adjusted according to the kind, content, and content ratio of the thermoplastic elastomer and liquid component.

In the patch of the present invention, risperidone can also be used in the form of a salt that can be absorbed transdermally. As such salt of risperidone, acid addition salt can be mentioned, and an addition salt of inorganic acid such as hydrohalic acid such as hydrochloric acid, hydrobromic acid and the like, sulfuric acid, nitric acid, phosphoric acid and the like, addition salt of organic acid such as monocarboxylic acid such as acetic acid, propionic acid, butyric acid and the like; hydroxycarboxylic acid such as hydroxyacetic acid, lactic acid, malic acid, citric acid and the like; oxocarboxylic acid such as pyruvic acid and the like; dicarboxylic acid such as oxalic acid, malonic acid, succinic acid, fumaric acid, maleic acid and the like; alkanesulfonic acid such as methanesulfonic acid, ethanesulfonic acid and the like; cyclohexanesulfamic acid; aromatic carboxylic acid such as benzenesulfonic acid, methylbenzenesulfonic acid, 2-hydroxybenzoic acid, 4-amino-2-hydroxybenzoic acid and the like, and the like can be mentioned. In the present invention, one or more kinds of the aforementioned salts can be selected and used.

In the patch of the present invention, risperidone in a free form is particularly preferably used from the aspect of easy availability, dispersibility in the adhesive layer and the like.

While the content of risperidone or a salt thereof in a patch is not particularly limited, it is preferably 0.1 wt %-10 wt %, particularly preferably 0.5 wt %-8 wt %, of the total weight of the adhesive layer, in consideration of the dispersibility in the adhesive layer and transdermal absorbability.

The adhesive layer forming the patch of the present invention may contain general additives as an optional component, such as excipient, dispersing agent, stabilizer, thickener, antioxidant, softening agent, flavoring agent, coloring agent, surfactant which is solid at ambient temperature and the like.

Examples of the excipient used in the present invention include silicon compound such as silicic anhydride, light anhydrous silicic acid, hydrous silicic acid and the like; cellulose derivative such as ethylcellulose, methylcellulose, hydroxypropylcellulose, hydroxypropylmethylcellulose and the like; synthetic water-soluble polymer such as polyvinyl alcohol and the like; aluminum compound such as dried aluminum hydroxide gel, hydrated aluminum silicate and the like; pigment such as kaolin, titanium oxide and the like, and the like.

Examples of the dispersing agent used in the present invention include gum arabic, alginic acid propyleneglycol ester, sodium dioctyl sulfosuccinate, lecithin and the like.

Examples of the stabilizer used in the present invention include stearic acid zinc salt, gelatin, dextran, povidone and the like.

Examples of the thickener used in the present invention include carboxyvinyl polymer, tragacanth and the like.

Examples of the antioxidant used in the present invention include dibutylhydroxytoluene, ascorbic acid, tocopherol, acetic acid tocopherol, butylhydroxyanisole, 2-mercaptobenzimidazole and the like.

Examples of the softening agent used in the present invention include polyisoprene rubber, polybutene and the like.

Examples of the flavoring agent used in the present invention include d-camphor, dl-camphor, cinnamaldehyde, peppermint oil, dl-menthol, l-menthol and the like.

Examples of the coloring agent used in the present invention include yellow iron oxide, yellow ferric oxide, carbon black and the like.

Examples of the surfactant which is solid at ambient temperature and used in the present invention include cholesterol, glycerolmonostearic acid ester, sorbitan monostearic acid ester and the like.

The patch of the present invention is prepared by extending adhesive layer with the above-mentioned constitution on a support.

In the present invention, the “support” is not particularly limited, and a conventional support can be used. For example, stretchable or non-stretchable woven fabric or non-woven fabric of polyethylene, polypropylene and the like, a film of polyethylene, polypropylene, polyester such as polyethylene terephthalate and the like, ethylene vinyl acetate copolymer, vinyl chloride and the like, or a foamed support of urethane, polyurethane and the like can be mentioned. These may be used alone, or a laminate of plural kinds thereof may be used. Furthermore, to prevent accumulation of static electricity on the support, the aforementioned woven fabric, non-woven fabric, film and the like constituting the support may contain an antistatic agent. Moreover, to achieve good anchor property to the adhesive layer, a non-woven fabric or woven fabric, or a laminate thereof with a film can be used as a support. The thickness of the support as a film is generally 10 μm-100 μm, preferably 15 μm-50 μm, and as a porous sheet such as woven fabric, non-woven fabric, foamed support and the like is generally 50 μm-2,000 μm, preferably 100 μm-1,000 μm.

In addition, the patch of the present invention can also be provided with a release liner generally used in the field of patch. As the release liner, glassine, polyethylene, polypropylene, polyester, polyethylene terephthalate, polystyrene, aluminum film, foamed polyethylene film, foamed polypropylene film and the like, or a laminate of two or more kinds of those mentioned above can be used, which may be silicone-treated or fluorine resin-treated, embossed and the like. The thickness of the release liner is generally 10 μm-200 μm, preferably 15 μm-150 μm.

The patch of the present invention can be produced, for example, by dissolving each of a thermoplastic elastomer and risperidone or a salt thereof in a liquid component, dissolving or dispersing same in a solvent such as toluene and the like, preparing a coating fluid for forming an adhesive layer, applying the obtained coating fluid on a support, and then drying same. When a release liner is used, it can be laminated by pressing a release liner on an adhesive layer. Alternatively, the aforementioned coating fluid may be applied on a release liner, dried to form an adhesive layer on a surface of the release liner, and thereafter the support may be press adhered on the adhesive layer. The coating fluid for forming an adhesive layer can be applied using a conventionally-used coater, such as a roll coater, a die coater, a gravure roll coater, a reverse roll coater, a kiss roll coater, a dip roll coater, a bar coater, a knife coater, a spray coater and the like. The aforementioned coating fluid is preferably dried under heating at, for example, about 40° C.-150° C. The adhesive layer after drying and containing risperidone or a salt thereof preferably shows 10 g/m²-1,000 g/m², more preferably 20 g/m²-800 g/m².

### EXAMPLES

The present invention is explained in more detail in the following by referring to Examples and Comparative Examples, which are not to be construed as limitative.

### Examples 1-3

### Preparation of Patch Containing Risperidone

According to the formulation shown in Table 1, each component constituting an adhesive layer was weighed. First, a styrene-isoprene-styrene block copolymer (“JSR SIS5002”, manufactured by JSR) was added to liquid paraffin (“KAYDOL”, manufactured by Sonneborn), and the mixture was dissolved in 60.0 parts by weight of toluene per 100 parts by weight of the total content of the adhesive layer components. Risperidone dissolved in other liquid component was added to the aforementioned solution, and the mixture was stirred to prepare a coating fluid to form the adhesive layer.

The above-mentioned coating fluid was applied to a silicone-treated polyethylene terephthalate (PET) film (release liner), and adjusted such that the weight of the adhesive layer after drying was 100 g/m². After drying for 30 min in an oven at 80° C., a polyethylene terephthalate (PET) film (support) was laminated on a surface of the adhesive layer, and cut into 15 cm×30 cm to give the object patch.

TABLE 1

| | content (wt %) | | |
|---|---|---|---|
| components | Example 1 | Example 2 | Example 3 |
| thermoplastic elastomer: | | | |
| styrene-isoprene-styrene | 16.0 | 16.0 | 16.0 |
| block copolymer | | | |
| liquid component: | | | |
| liquid paraffin | 48.0 | 32.0 | 44.0 |
| surfactant: | | | |
| sorbitan monolaurate | 1.6 | 1.8 | 1.8 |
| ester solvent: | | | |
| sebacic acid diethyl ester | 8.1 | 9.0 | 9.0 |
| middle chain fatty acid | | 12.0 | |
| triglyceride | | | |
| alcohol solvent: | | | |
| propylene glycol | | | 25.2 |
| amide solvent: | | | |
| N-methyl-2-pyrrolidone | 22.7 | 25.2 | |
| total | 80.4 | 80.0 | 80.0 |
| risperidone | 3.6 | 4.0 | 4.0 |

### Comparative Example 1

In the formulation of Example 3 in Table 1, a commercially available crosslinked carboxyl functional acrylic adhesive (“Durotak 87-2194”, manufactured by Henkel, solid content=40 wt %) was weighed, instead of the styrene-isoprene-styrene block copolymer, such that the solid content was the same as the thermoplastic elastomer content in Table 1, and liquid paraffin was added. Risperidone dissolved in other liquid component was added and the mixture was stirred to prepare a coating fluid to form an adhesive layer.

The coating fluid was applied to a silicone-treated PET-film (release liner), adjusted such that the weight of the adhesive layer after drying was 100 g/m², and dried for 60 min in an oven at 80° C. As a result, the coating was not cured and a patch could not be obtained.

### Example 4 and Comparative Example 2

### Preparation of Patch Containing Risperidone

According to the formulation shown in Table 2, an object patch was prepared in the same manner as in Examples 1-3. A pinene polymer (“YS resin PXN1150N”, manufactured by Yasuhara Chemical Co. Ltd.) was used as a tackifier for the patch of Comparative Example 2.

TABLE 2

content (wt %)

| | | Comparative |
|---|---|---|
| components | Example 4 | Example 2 |
| thermoplastic elastomer: | | |
| styrene-isoprene-styrene block | 16.0 | 16.0 |
| copolymer | | |
| liquid component: | | |
| liquid paraffin | 64.0 | 34.0 |
| surfactant: | | |
| sorbitan monolaurate | 1.0 | 1.0 |
| ester solvent: | | |
| sebacic acid diethyl ester | 4.7 | 4.7 |
| middle chain fatty acid | | |
| triglyceride | | |
| alcohol solvent: | | |
| propylene glycol | 13.3 | 13.3 |
| total | 83.0 | 53.0 |
| tackifier: | | |
| pinene polymer | | 30.0 |
| risperidone | 1.0 | 1.0 |

### Experimental Example 1

### In Vitro Skin Permeability Test

According to the method described in patent document 7, the skin extracted from the abdomen of male Wister rat (5-week-old) was set on a vertical Franz diffusion cell. Each patch of Examples 1-4 and Comparative Example 2 was punched out into a circular shape (diameter 1.0 cm) to give a sample, which was adhered to the skin of the rat in the diffusion cell (n=3). As a receptor layer, 10% by volume ethanol saline was used, and the risperidone content of the receptor solution was quantified over time by high performance liquid chromatography (HPLC). The HPLC qualification conditions are shown below.

### <HPLC Conditions>

HPLC system: high performance liquid chromatograph (LC2010C) manufactured by SHIMADZU CORPORATION

column: ODS, 4.6 mmφ×15 cm, 5 μm

column temperature: 40° C.

mobile layer: 0.1 wt % aqueous phosphoric acid solution/methanol/acetonitrile/SDS=4/1/5/0.01 (volume ratio)

detection wavelength: 254 nm

flow: 0.7 mL/min

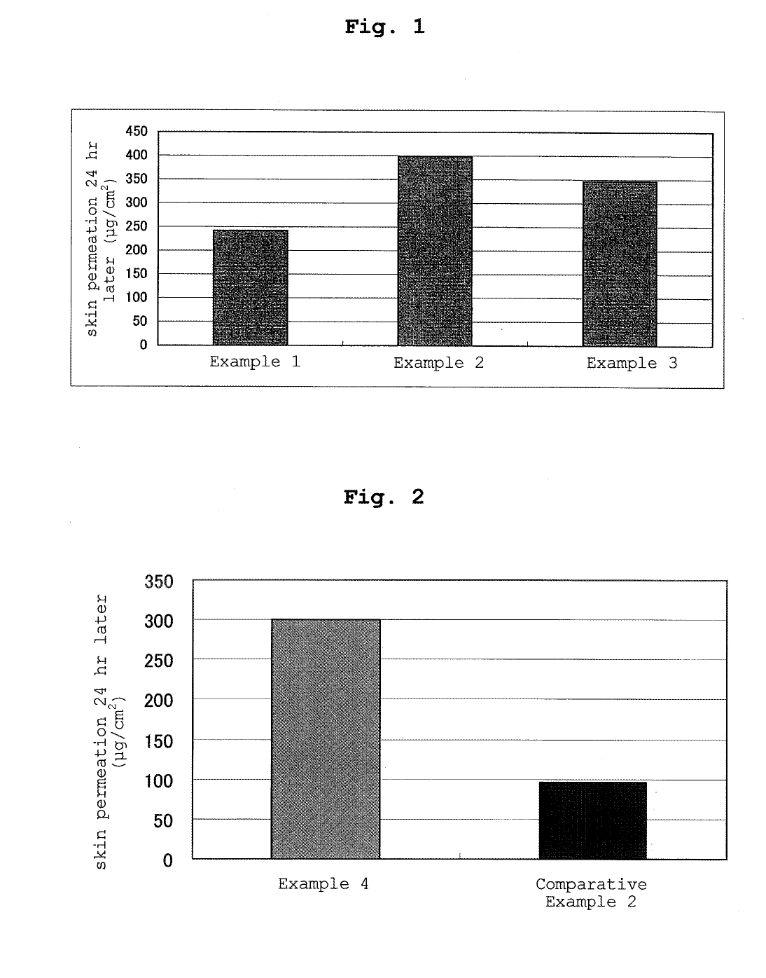

In the above-mentioned skin permeability test, the permeated amount of risperidone 24 hr after adhesion was measured and shown in FIGS. 1 and 2. From FIG. 1, it was shown that each patch of Examples 1-3 of the present invention is superior in the skin permeability of risperidone. Particularly, each patch of Examples 2 and 3, in which the liquid paraffin contents relative to the total amount of the liquid component were 40 wt % and 55 wt %, showed good skin permeability.

From FIG. 2, moreover, it was shown that the patch of Example 4 of the present invention showed good skin permeability as compared to the patch of Comparative Example 2 containing 30 wt % of a tackifier.

### Experimental Example 2

### In Vivo Transdermal Absorbability Test

The patch of Example 3 was cut into 4 cm×6 cm to give a sample, which was adhered to the back of five male slc/HWY hairless rats (7-week-old). At 3, 6, 9, 24, 48 and 72 hr after adhesion, blood samples were collected from the cervical vein and centrifuged to give plasma. The concentration of risperidone in the obtained plasma was measured by liquid chromatography-tandem mass analysis method (LC-MS/MS). The measurement conditions of LC-MS/MS are as follows.

### <HPLC Conditions>

HPLC system: high performance liquid chromatograph (1200 Series) manufactured by Agilent Technologies

analysis column: Atlantis dc18, 2.1 mm I.D.×150 mm, 5 μm

column temperature: 40° C.

mobile layer: methanol/0.05 wt % formic acid solution=1/1 (volume ratio)

flow: 0.2 mL/min

### <MS/MS Conditions>

Tandem mass spectrometer: API4000, manufactured by AB Sciex Pte

interface: Turbo-V spray

ionization method: ESI, plus ion mode

measurement ion: risperidone 422.6 191.4

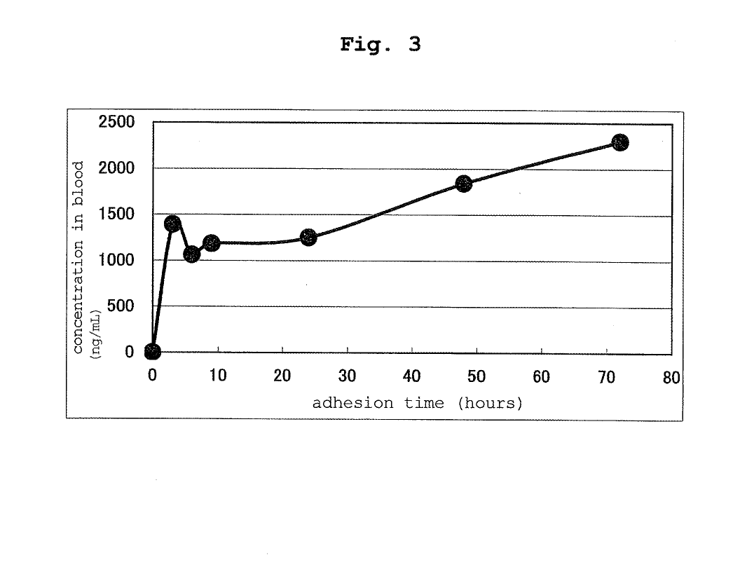

The results of the above-mentioned transdermal absorbability test are shown in FIG. 3. From FIG. 3, it was shown that risperidone was transdermally absorbed rapidly after adhesion to the skin, and transferred into the blood. During the period of continuous adhesion for 3 days, the concentration of risperidone in plasma was maintained at about 1,000 ng/mL on average. It was shown that risperidone in the patch of the present invention is transdermally absorbed in a sustained manner for a long time.

### Experimental Example 3

### Primary Skin Irritation Test

The patch of Example 3 was cut into 2.5 cm×2.5 cm to give a sample, which was occlusively adhered to the back, from which the hair was removed with an electric clipper, of a male white house rabbit (Kbs:NZW) for 24 hr. After lapse of 1, 24, 48 and 72 hr from the peeling off of the sample, the skin condition was evaluated by the method of Draize. J. H. et al. (see J. Pharmacol. Exp. Ther. 82 377-390), and the primary irritation index value (P.I.I. value) was calculated. As a result, the P.I.I. value was 1.0, which means an evaluation acknowledging the presence of a weak skin irritation. It was clarified that the patch shows skin irritation of a low level.

### INDUSTRIAL APPLICABILITY

According to the present invention, a patch showing sufficient adhesiveness on adhesion to the skin, reduced skin irritation, good skin permeability of risperidone or a salt thereof, and superior transdermal absorbability can be provided. Therefore, a preparation containing risperidone or a salt thereof, which can be utilized as a new therapeutic drug for schizophrenia for administration by a route other than oral administration, can be provided.

This application is based on a patent application No. 2011-179988 filed in Japan, the contents of which are incorporated in full herein.

1. A patch comprising an adhesive layer retaining a drug formed on a support, wherein the aforementioned adhesive layer comprises a thermoplastic elastomer, a liquid component in an amount exceeding 300 parts by weight relative to 100 parts by weight of the elastomer, and risperidone or a salt thereof, and optionally comprises a tackifier, wherein the content thereof in the adhesive layer is not more than 10 wt %.

2. The patch according to claim 1, wherein the liquid component comprises one or more kinds selected from the group consisting of a non-volatile hydrocarbon oil, a liquid surfactant, an ester solvent, an alcohol solvent and an amide solvent.

3. The patch according to claim 1, wherein the liquid component comprises a non-volatile hydrocarbon oil and a liquid component other than the non-volatile hydrocarbon oil.

4. The patch according to claim 3, wherein the non-volatile hydrocarbon oil is liquid paraffin.

5. The patch according to claim 3, wherein the liquid component other than the non-volatile hydrocarbon oil comprises one or more kinds selected from the group consisting of a liquid surfactant, an ester solvent, an alcohol solvent and an amide solvent.

6. The patch according to claim 3, wherein the liquid component other than the non-volatile hydrocarbon oil comprises one or more kinds of ester solvents, and one or more kinds selected from the group consisting of an alcohol solvent and an amide solvent.

7. The patch according to claim 2, wherein the liquid surfactant is a liquid sorbitan fatty acid ester.

8. The patch according to claim 4, wherein the content of the liquid component in the adhesive layer is not less than 60 wt %.

9. The patch according to claim 4, wherein the thermoplastic elastomer is a styrene block copolymer.

10. The patch according to claim 9, wherein the styrene block copolymer is one or more kinds selected from the group consisting of a styrene-isoprene-styrene block copolymer and a styrene-isoprene block copolymer.

11. The patch according to claim 4, wherein the adhesive layer is free of a tackifier.

12. The patch according to claim 2, wherein the non-volatile hydrocarbon oil is liquid paraffin.

13. The patch according to claim 4, wherein the liquid surfactant is a liquid sorbitan fatty acid ester.

14. The patch according to claim 5, wherein the content of the liquid component in the adhesive layer is not less than 60 wt %.

15. The patch according to claim 5, wherein the thermoplastic elastomer is a styrene block copolymer.

16. The patch according to claim 15, wherein the styrene block copolymer is one or more kinds selected from the group consisting of a styrene-isoprene-styrene block copolymer and a styrene-isoprene block copolymer.

17. The patch according to claim 5, wherein the adhesive layer is free of a tackifier.
